(19) United States
(12) Patent Application Publication
(10) Pub. No.: US 2025/0058096 A1
(43) Pub. Date: Feb. 20, 2025

(54) SKIN DRUG INJECTOR

(71) Applicants: Hyun Mee NOH, Yongin-si (KR); NOBAMEDI CO., LTD., Yongin-si (KR)

(72) Inventor: Hyun Mee NOH, Yongin-si (KR)

(73) Assignees: Hyun Mee NOH, Yongin-si (KR); NOBAMEDI CO., LTD., Yongin-si (KR)

(21) Appl. No.: 18/670,421

(22) Filed: May 21, 2024

(30) Foreign Application Priority Data
Aug. 18, 2023 (KR) 10-2023-0108365

(51) Int. Cl. A61M 37/00

(52) U.S. Cl. CPC A61M 37/0015; A61M 2037/0023

(57) ABSTRACT

Provided is a skin drug injector connected to a drug bottle that is filled with a drug and that includes an opening portion, in which the skin drug injector includes a connection portion comprising a drug discharge hole connected to the opening portion of the drug bottle and allowing a drug to pass through, an inner insertion port tightly coupled to an inner circumferential surface of the opening portion of the drug bottle, an insertion groove formed inside the inner insertion port, and a fitting opening communicating with the insertion groove, a body portion comprising a fitting rod portion coupled to the insertion groove of the connection portion and including a drug passage hole corresponding to the drug discharge hole, an enlarged diameter portion formed on a lower portion of the fitting rod portion, and a chamber to be filled with the drug, and an injection portion connected to an opening of the enlarged diameter portion of the body portion and including and micro-needles N passed through the drug discharge hole and exposed outside, in which opening rates of the drug passage hole of the body portion and the drug discharge hole of the connection portion are adjusted so as to adjust an amount of drug injection.

### CROSS-REFERENCE TO RELATED APPLICATION

This application claims priority to Korean Patent Applications No. 10-2023-0108365, filed in the Korean Intellectual Property Office on Aug. 18, 2023, the entire contents of which are hereby incorporated by reference.

### BACKGROUND

### Technical Field

The present disclosure relates to a skin drug injector, and more particularly, to a skin drug injector that induces rapid absorption of drugs into the skin after skin perforation.

### Description of Related Art

Recently, interest in skin care has been fast rising and various forms of skin treatment methods are tried for treating skin conditions such as acne treatment, pore reduction, wrinkle removal, scar removal, and the like.

Delivery of cosmetics or drugs through the skin has a very low absorption rate due to the difficulty of drugs penetrating the stratum corneum.

Korean Patent No. 10-1492732 describes “Skin Treatment Apparatus” which perforates the skin with a plurality of micro-needles while simultaneously applying drugs to the skin to thus enhance the absorption effect of drugs.

While the related technology that simultaneously performs skin perforation and drug application to the skin offers the advantages of convenience and time saving of procedure, there is a problem that the use of mechanical elements such as valves or springs to open and close a large drug injection hole formed in the center of the micro-needle module causes disadvantages such as complicated structure of the module, increased manufacturing cost, and cumbersome assembly.

### SUMMARY

The present disclosure has been proposed to solve the problems of the related art, and an object of the present disclosure is to provide a skin drug injector that performs both skin perforation and drug application on skin simultaneously, while adjusting the opening rate of a drug injection hole through which the drug is passed so as to adjust an amount of drug injection.

Another object of the present disclosure is to provide a skin drug injector that prevents loss of drug and injects the drug into skin effectively.

In order to achieve the above and other objects of the present disclosure, a skin drug injector is provided, which may be connected to a drug bottle that is filled with a drug and that includes an opening portion, in which the skin drug injector may include a connection portion comprising a drug discharge hole connected to the opening portion of the drug bottle and allowing a drug to pass through, an inner insertion port tightly coupled to an inner circumferential surface of the opening portion of the drug bottle, an insertion groove formed inside the inner insertion port, and a fitting opening communicating with the insertion groove, a body portion comprising a fitting rod portion coupled to the insertion groove of the connection portion and including a drug passage hole corresponding to the drug discharge hole, an enlarged diameter portion formed on a lower portion of the fitting rod portion, and a chamber to be filled with the drug, and an injection portion connected to an opening of the enlarged diameter portion of the body portion and including and micro-needles N passed through the drug discharge hole and exposed outside, in which opening rates of the drug passage hole of the body portion and the drug discharge hole of the connection portion may be adjusted so as to adjust an amount of drug injection.

The drug discharge hole may include a circular hole having a diameter equal to or greater than the drug passage hole, and an arc-shaped hole communicating with, and bent from the circular hole and tapered in width towards an end.

The connection portion may include a fitting opening communicating with the insertion groove, and the fitting opening may include an arc-shaped guard formed on one side of an inner circumferential surface, the body portion may include a protrusion protruding from an outer circumferential surface of the fitting rod portion, and the protrusion may be inserted into an inner circumferential surface of the fitting opening to guide a rotation of the body portion within a range except for the guard.

The injection portion may include a container including a plurality of drug discharge holes formed in a bottom, and stepped portions protruding externally from the bottom of the container to correspond to a plurality of drug discharge holes, and micro-needles formed on lower portions of the stepped portions.

The micro-needles may be formed integrally with the stepped portions, in which the micro-needles may be formed at lower portions of the stepped portions, and the stepped portions may be configured to surround the drug discharge hole to form a partition wall, and include a groove formed inside the partition wall to keep the drug therein.

According to an embodiment, a micro-needle block may be fastened inside the container, the micro-needle block may include a plurality of guide blocks including both sides elongated in a longitudinal direction, drug transfer grooves formed on the elongated sides and communicating with the plurality of drug discharge holes, and body portion insertion grooves formed in the both sides, and a plate-shaped micro-needle portion including a plate-shaped body portion fastened with the body portion insertion grooves of the guide block, and a plurality of micro-needles extending from the plate-shaped body portion, in which upon engagement of the guide block, the plate-shaped body portion may be fitted in the body portion insertion grooves and fastened with the plate-shaped micro-needle portion, and the drug transfer grooves may be engaged with each other to form drug transfer holes, each of the stepped portions may house micro-needles therein, communicate with the drug discharge holes, and protrude externally from the bottom of the container, the micro-needles may be passed through the stepped portions to be exposed from lower portions of the stepped portions, and the drug may be discharged through the drug transfer holes to the drug discharge holes along a surface of the micro-needles.

According to another example, the injection portion may include a container including a plurality of drug discharge holes formed in a bottom, and a micro-needle block fastened inside the container, and the micro-needle block may include a plurality of guide blocks including both sides elongated in a longitudinal direction, drug transfer grooves formed on the both sides and communicating with the plurality of drug discharge holes, body portion insertion grooves formed in the both sides, and a plurality of block protrusions passed through the drug discharge holes to protrude out of the bottom of the container, and a plate-shaped micro-needle portion including a plate-shaped body portion fastened with the body portion insertion grooves of the guide block, and a plurality of micro-needles extending from the plate-shaped body portion, in which upon engagement of the guide block, the plate-shaped body portion may be fitted in the body portion insertion grooves and fastened with the plate-shaped micro-needle portion, the micro-needles N may be housed in the block protrusions, respectively, the drug transfer grooves may be engaged with each other to form drug transfer holes, the micro-needles may be passed through the block protrusions to be exposed from the lower portion, and the drug may be discharged through the drug transfer holes along the surface of the micro-needles housed inside the block protrusions.

The body portion may include an inlet pipe connected to the drug passage hole and protruding into the chamber, and the inlet pipe may allow the drug in the drug bottle to flow into the chamber of the body portion through the drug passage hole.

The body portion may include a bent ledge formed on an inner side of the enlarged diameter portion, the opening of the enlarged diameter portion may be tapered from an end portion toward the bent ledge, and the container may include a protruding end corresponding to the bent ledge of the enlarged diameter portion.

According to the examples of the present disclosure, there is an effect that, while skin perforation and drug application on skin are performed simultaneously, it is possible to adjust the opening rate of the drug injection hole through which the drug passes, thereby adjusting the amount of drug injection.

Further, according to the examples of the present disclosure, the loss of drug can ay be prevented and the drug can be injected into the skin effectively.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1 is a perspective view of a skin drug injector according to an example of the present disclosure;

FIG. 2 is an exploded perspective view of a skin drug injector according to an example of the present disclosure;

FIG. 3 is a cross-sectional perspective view of a skin drug injector according to an example of the present disclosure;

FIG. 4 is an exploded cross-sectional view of a skin drug injector according to an example of the present disclosure;

FIG. 5 is an exploded perspective view of a body portion and a connection portion of a skin drug injector viewed from below according to an example of the present disclosure;

FIGS. 6A to 6C are perspective views showing steps of adjusting an opening rate of a connection portion and a body portion of a skin drug injector viewed from below according to an example of the present disclosure;

FIGS. 7A to 7C are bottom views corresponding to the respective adjustment steps in FIGS. 6A to 6C;

FIGS. 8A to 8C are plan views showing steps of adjusting an opening rate of a connection portion and a body portion of a skin drug injector viewed from above according to an example of the present disclosure;

FIG. 9 is a perspective view of an injection portion of a skin drug injector according to an example of the present disclosure;

FIG. 10 is a perspective view showing an injection portion of a skin drug injector according to another example the present disclosure;

FIG. 11 is an exploded perspective view of the injection portion in FIG. 10; and

FIG. 12 is an exploded perspective view of an injection portion of a skin drug injector according to still another example of the present disclosure.

### DETAILED DESCRIPTION

Hereinafter, certain preferred examples of the disclosure will be described in detail below based on the attached drawings.

The examples to be described below are intended to explain the present disclosure in detail such that a person skilled in the art can easily practice the present disclosure, and do not mean that the technical idea and scope of the disclosure are limited.

In addition, it is to be noted that the size or shape of the components shown in the drawings may be exaggerated for clarity and convenience of explanation, and terms specifically defined in consideration of the configuration and operation of the present disclosure may vary according to the intention or custom of the user or operator, and these terms should be defined based on the content throughout this specification.

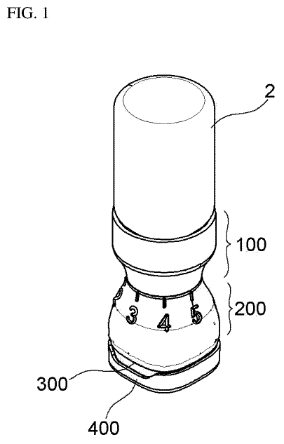

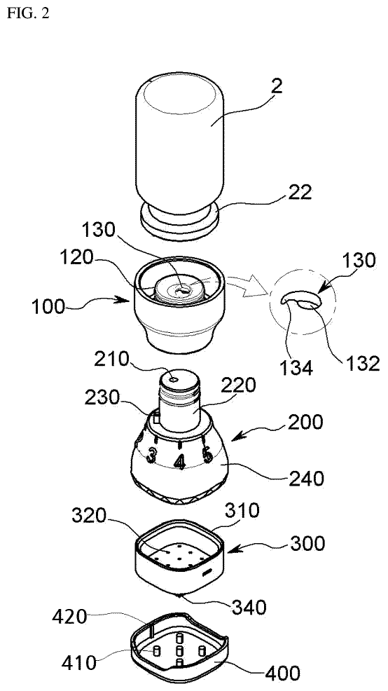

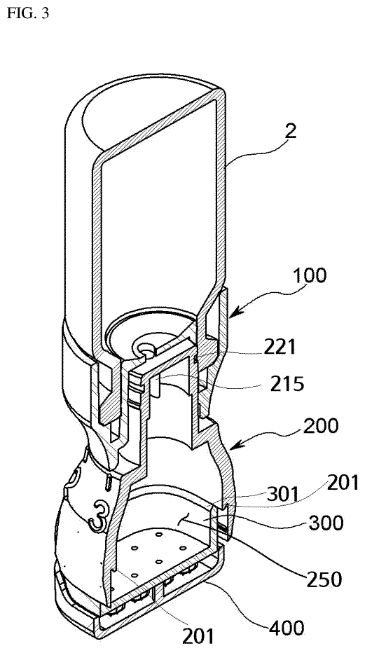

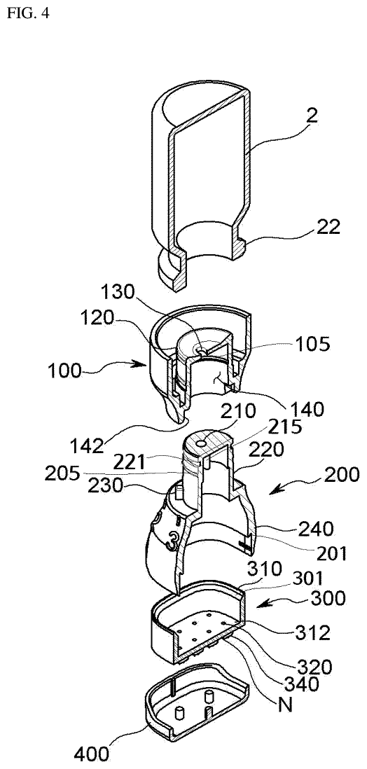

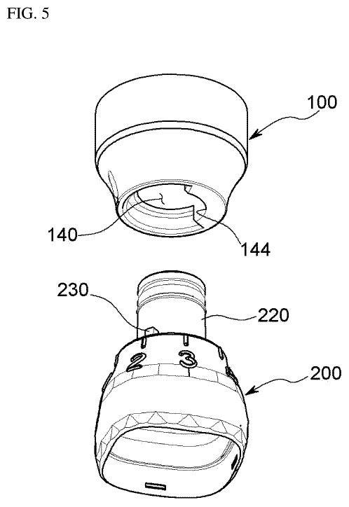

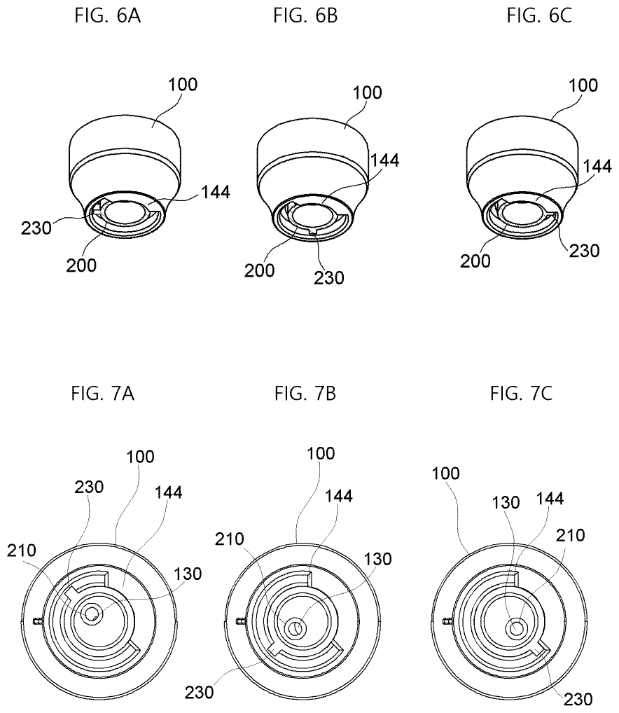

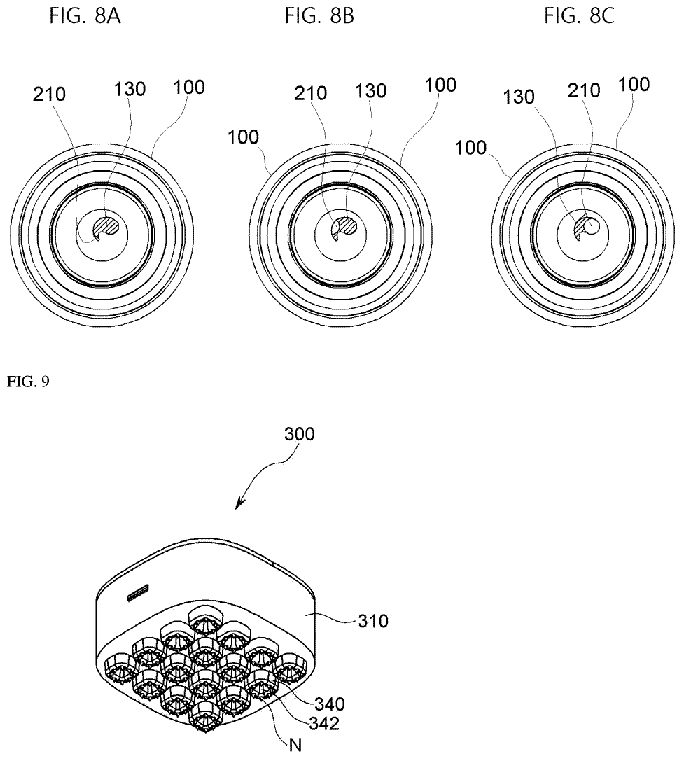

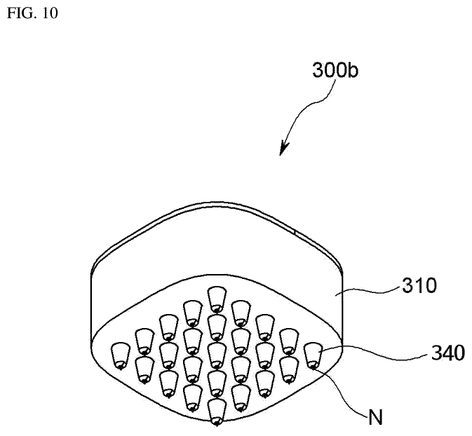

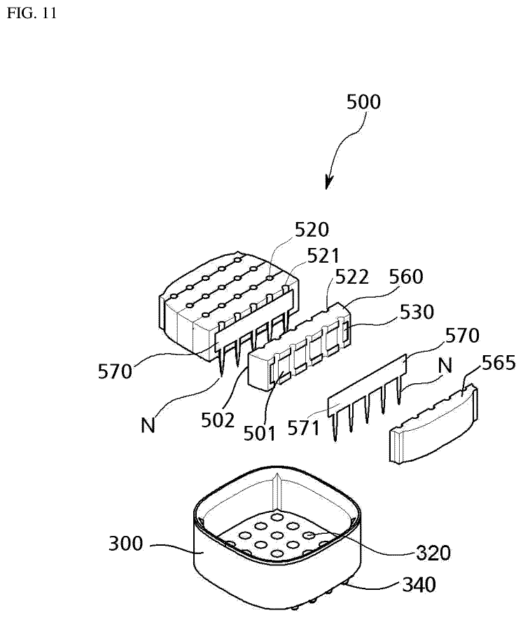

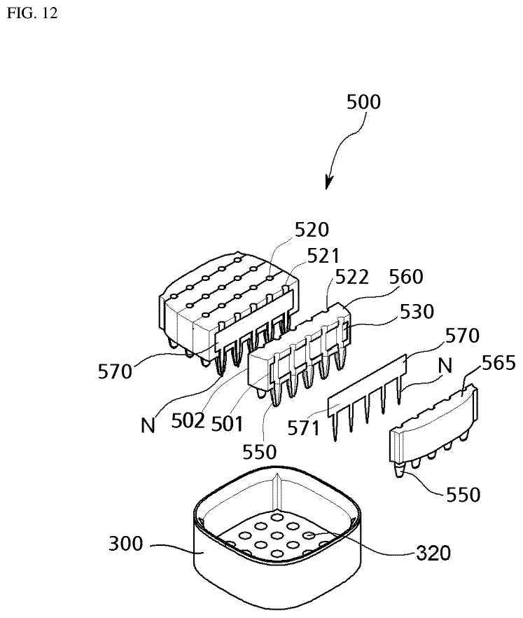

In the accompanying drawings, FIG. 1 is a perspective view of a skin drug injector according to an example of the present disclosure, FIG. 2 is an exploded perspective view of a skin drug injector according to an example of the present disclosure, FIG. 3 is a cross-sectional perspective view of a skin drug injector according to an example of the present disclosure, FIG. 4 is an exploded cross-sectional view of a skin drug injector according to an example of the present disclosure, FIG. 5 is an exploded perspective view of a body portion and a connection portion of a skin drug injector viewed from below according to an example of the present disclosure, FIGS. 6A to 6C are perspective views showing steps of adjusting an opening rate of a connection portion and a body portion of a skin drug injector viewed from below according to an example of the present disclosure, FIGS. 7A to 7C are bottom views corresponding to the respective adjustment steps in FIGS. 6A to 6C, FIGS. 8A to 8C are plan views showing steps of adjusting an opening rate of a connection portion and a body portion of a skin drug injector viewed from above according to an example of the present disclosure, FIG. 9 is a perspective view of an injection portion of a skin drug injector according to an example of the present disclosure, FIG. 10 is a perspective view showing an injection portion of a skin drug injector according to another example the present disclosure, FIG. 11 is an exploded perspective view of the injection portion in FIG. 10, and FIG. 12 is an exploded perspective view of an injection portion of a skin drug injector according to still another example of the present disclosure.

Referring to FIGS. 1 to 4, a skin drug injector according to an example may be connected to a drug bottle 2 that is filled with a drug L inside and that includes an opening portion 22 formed thereon, and may include:
a connection portion 100 including a drug discharge hole 130 coupled to the opening portion 22 of the drug bottle 2 and allowing the drug L to pass through, an inner insertion port 120 tightly coupled to an inner circumferential surface of the opening portion 22 of the drug bottle 2, an insertion groove 140 formed inside the inner insertion port 120, and a fitting opening 142 communicating with the insertion groove 140;
a body portion 200 including a fitting rod portion 220 coupled to the insertion groove 140 of the connection portion 100 and including a drug passage hole 210 corresponding to the drug discharge hole 130, an enlarged diameter portion 240 formed on a lower portion of the fitting rod portion 220, and a chamber 250 filled with the drug L; and
an injection portion 300 coupled to an opening of the enlarged diameter portion 240 of the body portion 200 and including a container 310 having a plurality of drug discharge holes 320 formed in a bottom, and micro-needles N that are exposed outside,
in which opening rates of the drug passage hole 210 of the body portion 200 and the drug discharge hole 130 of the connection portion 100 are adjusted so as to adjust an amount of drug injection.

The drug bottle 2 is attachable to and detachable from the connection portion 100, and fit into an upper portion of the connection portion 100 with the opening portion facing downward.

The connection portion 100 has the inner insertion port 120 formed inside and is spaced apart from an outer wall such that the opening portion 22 of the drug bottle 2 may be inserted into the space.

For the secure fastening of the connection portion 100 and the body portion 200, the connection portion 100 includes a fitting groove 105 formed on an inner surface of the inner insertion port 120, and the body portion 200 includes a fitting protrusion 205 corresponding to the fitting groove, which is on an outer surface of the fitting rod portion 220, such that the fitting protrusion 205 is force fit in the fitting groove 105. Accordingly, once the connection portion 100 and the body portion 200 are fit, they are hardly disconnected.

The fitting rod portion 200 of the body portion 200 includes one or more recesses 221, and a friction member such as an O-ring is fitted into the recesses 221. Therefore, the connection portion 100 and the body portion 200 are kept in tight contact with each other while rotating relative to each other so that the leakage of the drug L can be prevented.

A cap 400 is further provided, which is coupled to the injection portion 300 to protect the micro-needles. The cap 400 is brought into tight contact with the outer surface of the container 310 of the injection portion 300 and fastened therein, and one or more fitting protrusions 420 are formed on the inner circumferential surface of the cap 400. Due to the presence of the fitting protrusions 420, the cap 400 is securely fastened with the injection portion 300, thus preventing the cap 400 from being disconnected. Meanwhile, in the fastened state, the bottom surface of the cap 400 is spaced apart from ends of the micro-needles N formed in stepped portions 340 of the injection portion 300 so as to protect the micro-needles N. The cap 400 includes one or more cap protrusions 410 formed therein, in which the cap protrusions 410 protrude upward from a bottom. The cap protrusions 410 are positioned in a space defined in the stepped portions 340. Even when an external force is applied to the cap 400, the cap protrusions 410 may prevent damage to the micro-needles N.

Referring to FIG. 2, the drug discharge hole 130 for the drug to pass through is formed in a top surface of the inner insertion port 120. The drug discharge hole 130 includes a circular hole 132 having a diameter equal to or greater than the drug passage hole 210, and an arc-shaped hole 134 communicating with the circular hole 132 and bent and tapered in width towards the end. Because the drug discharge hole 130 is of varying size, a hole of varying size may be formed by overlapping the drug discharge hole 130 with the drug passage hole 210, and it is possible to adjust an amount of the drug L discharge by adjusting the opening rate with the hole of varying size.

Further, although not shown, the arc-shaped hole 134 may further include a plurality of auxiliary circular holes (not shown) and may also include a plurality of auxiliary circular holes with different diameters, from large to small.

Referring to FIG. 5, an arc-shaped guard 144 is formed on one side of the inner circumferential surface of the fitting opening 142 of the connection portion 100. A protrusion 230 corresponding to the arc-shaped guard 144 is provided, which may protrude from an outer circumferential surface of the fitting rod portion 220 of the body portion 200.

In other words, the fitting rod portion 220 is vertically formed on an upper end of the enlarged diameter portion 240 of the body portion 200, with a smaller diameter than the upper end of the enlarged diameter portion 240, and the protrusion 230 is formed between the upper end of the enlarged diameter portion 240 and a lower end of the fitting rod portion 220. Therefore, the protrusion 230 may be inserted into the inner circumferential surface of the fitting opening 142 of the connection portion 100 and kept in tight contact, and may guide the rotation of the body portion 200 within an angular range excluding the guard 144, when the body portion 200 is rotated.

The opening rate, which is a degree by which the drug passage hole 210 of the body portion 200 and the drug discharge hole 130 of the connection portion 100 are aligned or misaligned, may be adjusted by the rotation of the body portion 200, and by this adjustment, it is possible to adjust the amount of drug injection passed therethrough. The opening rate is adjusted by the relative rotation of the body portion 200 and the connection portion 100, and either the body portion 200 or the connection portion 100 is rotated.

This will be described by referring to FIGS. 6A to 8C.

As shown in FIGS. 6A to 8A, by rotating the body portion 200 to make an end portion of the arc-shaped hole 134 of the drug discharge hole 130 misaligned with the drug passage hole 210, the drug flow may be minimized or blocked.

Then, as shown in FIGS. 6 to 8B, by rotating the body portion 200 to make the middle portion of the arc-shaped hole 134 of the drug discharge hole 130 aligned with the drug passage hole 210, the drug flow may increase.

Then, as shown in FIG. 6 to FIG. 8C, by further rotating the body portion 200 to make the circular hole 132 of the drug discharge hole 130 aligned with the drug passage hole 210, the drug flow may be maximized.

The body portion 200 includes an inlet pipe 215 connected to the drug passage hole 210 and protruding into the chamber 250, and the drug L in the drug bottle 2 flows through the drug passage hole 210 and the inlet pipe 215 into the chamber 250 of the body portion 200. The inlet pipe 215 allows the drug L to pass through the connection portion 100 and flow into the chamber 250 stably.

The injection portion 300 is coupled to the opening of the enlarged diameter portion 240 of the body portion 200, and includes a container 310 having a plurality of drug discharge holes 320 formed in the bottom, and micro-needles N that are exposed outside.

The body portion 200 includes a bent ledge 201 formed on an inner side of the enlarged diameter portion 240, and the opening of the enlarged diameter portion 240 is tapered from an end portion toward the bent ledge 201. The container 310 of the injection portion 300 includes a corresponding protruding end 301. The protruding end 301 is formed in a shape corresponding to the shape of the bent ledge 201 of the enlarged diameter portion 240, and the body portion 200 is securely fastened with the injection portion 300 so that the drug L is stably contained.

According to an example, as shown in FIG. 9, the injection portion 300 includes the container 310 having a plurality of drug discharge holes 320 formed in the bottom, the stepped portions 340 protruding externally from the bottom of the container 310 so as to correspond to the plurality of drug discharge holes 320, and the micro-needles N formed on the lower portion of the stepped portions 340, in which the micro-needles N are integrally formed with the stepped portions 340 on the lower portion of the stepped portions 340, the stepped portions 340 are configured to surround the drug discharge holes 320 to form partition walls, and there are grooves 342 formed inside the partition walls so that the drug may stay therein.

Because the stepped portions 340 have a surface that comes into contact with the skin, when the micro-needles N are inserted into the skin, the stepped portions apply pressure to the skin to facilitate the insertion of the micro-needles N into the skin.

The micro-needles N protrude from the ends of the stepped portions 340, and the micro-needles N pierce the epidermis of the skin to create perforations, through which the drug L is injected.

The stepped portions 340 have the grooves 342 formed therein to surround the drug discharge holes 320. As shown, the protrusions may be rectangular in shape, or may be circular, triangular, or other shapes. The protrusions are not particularly limited in shapes, as long as they have the partition walls to surround the drug discharge holes 320, and the grooves 342 formed inside the partition walls to keep the drug held therein.

The drug supplied from the drug discharge holes 320 may be applied on the surfaces of the micro-needles N and injected into the skin along the micro-needles N through the skin perforations formed by the micro-needles N. That is, the drug is supplied to the spaces surrounded by the stepped portions 340, evenly applied on the micro-needles N, and flows, along with the micro-needles N, into the skin through the perforations formed on the skin.

A skin drug injector according to another example is shown in FIGS. 10 to 12.

As shown in FIGS. 10 to 11, according to another example of the present disclosure, the injection portion 300 includes a container 310 including a plurality of drug discharge holes 320 formed in the bottom, stepped portions 340 protruding externally from the bottom of the container 310 to correspond to the plurality of drug discharge holes 320, and micro-needles N formed on lower portions of the stepped portions 340.

A micro-needle block 500 is fastened inside the container 310, and the micro-needle block 500 includes: a plurality of guide blocks 560 including both sides 501, 502 elongated in a longitudinal direction, drug transfer grooves 521, 522 formed on the elongated sides 501, 502 and communicating with a plurality of drug discharge holes 320, and body portion insertion grooves 530 formed in both sides 501, 502; and a plate-shaped micro-needle portion 570 including a plate-shaped body portion 571 fastened with the body portion insertion grooves 530 of the guide block 560 and a plurality of micro-needles N extending from the plate-shaped body portion 571.

Upon engagement of the guide block 560, the plate-shaped body portion 571 is fitted in the inner body portion insertion grooves 530 and fastened with the plate-shaped micro-needle portion 570, and the drug transfer grooves 521, 522 are engaged with each other to form drug transfer holes 520.

Each of the stepped portions 340 houses micro-needles N therein, communicates with the drug discharge holes 320, and protrudes externally from the bottom of the container 310.

The micro-needles N are passed through the stepped portions 340 to be exposed from the lower portions of the stepped portions 340, and the drug L is discharged through the drug transfer holes 520 to the drug discharge holes 320 along the surface of the micro-needles N.

The micro-needle block 500 may have the drug transfer grooves 521 only on one of the two sides and may have an outer guide block 565 contacting the inner surface of the container 310 on the other side. Meanwhile, a groove may be formed on a corner portion of the container 310 of the injection portion 300 to increase the coupling area and thus secure coupling, and a coupling protrusion may be formed on a corner of the outer guide block 565 in a shape corresponding to the shape of the corner portion of the container 310.

The injection portion 300 is formed of a synthetic resin material, and the plate-shaped micro-needle portion 370 is formed of a metal material.

The micro-needles N are positioned in the plurality of stepped portions 340, and the drug L is supplied into the stepped portions 340 through the drug discharge holes 320, such that the drug L is applied on the micro-needles N in the stepped portions and flow downward along the micro-needles N. Accordingly, the drug L applied on the surface of the micro-needles N and the drug L flowing down along the micro-needles N may be introduced into the skin as the micro-needles form perforations in the skin.

According to still another example of the present disclosure, as shown in FIG. 12, the injection portion 300 includes a container 310 having a plurality of drug discharge holes 320 formed in a bottom, and a micro-needle block 500 fastened inside the container 310.

The micro-needle block 500 includes: a plurality of guide blocks 560 including both sides 501, 502 elongated in a longitudinal direction, drug transfer grooves 521, 522 formed on both sides 501, 502 and communicating with a plurality of drug discharge holes 320, body portion insertion grooves 530 formed in both sides 501, 502, and a plurality of block protrusions 550 passed through the drug discharge holes 320 to protrude out of the bottom of the container 310; and
a plate-shaped micro-needle portion 570 including a plate-shaped body portion 571 fastened with the body portion insertion grooves 530 of the guide block 560, and a plurality of micro-needles N extending from the plate-shaped body portion 571.

Upon engagement of the guide block 560, the plate-shaped body portion 571 is fitted in the inner body portion insertion grooves 530 and fastened with the plate-shaped micro-needle portion 570, the micro-needles N are housed in the block protrusions 550, respectively, and the drug transfer grooves 521, 522 are engaged with each other to form the drug transfer holes 520.

The micro-needles N are passed through the block protrusions 550 to be exposed from the lower portion, and the drug L is discharged through the drug transfer holes 520 along the surface of the micro-needles N housed inside the block protrusions 550.

The injection portion 300 is formed of a synthetic resin material, and the plate-shaped micro-needle portion 370 is formed of a metal material.

The micro-needles N are positioned in the plurality of block protrusions 550, and drug L is supplied into the block protrusions 550 through the drug discharge holes 520, applied on the micro-needles N in the protrusions, and flows downward along the micro-needles N. Accordingly, the drug L applied on the surface of the micro-needles N and the drug L flowing down along the micro-needles N may be introduced into the skin as the micro-needles form perforations in the skin.

Although the description has been made in relation to certain embodiments, it will be readily apparent to those skilled in the art that various modifications and variations can be made without departing from the gist and scope of the disclosure, and it is obvious that all such changes and modifications fall within the scope of the appended claims.

1. A skin drug injector connected to a drug bottle that is filled with a drug and that includes an opening portion, the skin drug injector comprising:
a connection portion comprising a drug discharge hole connected to the opening portion of the drug bottle and allowing a drug to pass through, an inner insertion port tightly coupled to an inner circumferential surface of the opening portion of the drug bottle, an insertion groove formed inside the inner insertion port, and a fitting opening communicating with the insertion groove;
a body portion comprising a fitting rod portion coupled to the insertion groove of the connection portion and including a drug passage hole corresponding to the drug discharge hole, an enlarged diameter portion formed on a lower portion of the fitting rod portion, and a chamber to be filled with the drug; and
an injection portion connected to an opening of the enlarged diameter portion of the body portion and including and micro-needles N passed through the drug discharge hole and exposed outside, wherein
opening rates of the drug passage hole of the body portion and the drug discharge hole of the connection portion are adjusted so as to adjust an amount of drug injection.

2. The skin drug injector according to claim 1, wherein the drug discharge hole includes
a circular hole having a diameter equal to or greater than the drug passage hole, and
an arc-shaped hole communicating with, and bent from the circular hole and tapered in width towards an end.

3. The skin drug injector according to claim 2, wherein the arc-shaped hole further includes a plurality of auxiliary circular holes and the plurality of auxiliary circular holes have different diameters, respectively.

4. The skin drug injector according to claim 1, wherein the connection portion includes a fitting opening communicating with the insertion groove, and the fitting opening includes an arc-shaped guard formed on one side of an inner circumferential surface,
the body portion includes a protrusion protruding from an outer circumferential surface of the fitting rod portion, and
the protrusion is inserted into an inner circumferential surface of the fitting opening to guide a rotation of the body portion within a range except for the guard.

5. The skin drug injector according to claim 1, wherein the injection portion includes:
a container including a plurality of drug discharge holes formed in a bottom;
stepped portions protruding externally from the bottom of the container to correspond to a plurality of drug discharge holes; and
micro-needles formed on lower portions of the stepped portions.

6. The skin drug injector according to claim 5, wherein the micro-needles are formed integrally with the stepped portions, the micro-needles being formed at lower portions of the stepped portions, and
the stepped portions are configured to surround the drug discharge hole to form a partition wall, and include a groove formed inside the partition wall to keep the drug therein.

7. The skin drug injector according to claim 5, wherein a micro-needle block is fastened inside the container,
the micro-needle block includes:
a plurality of guide blocks including both sides elongated in a longitudinal direction, drug transfer grooves formed on the elongated sides and communicating with the plurality of drug discharge holes, and body portion insertion grooves formed in the both sides; and
a plate-shaped micro-needle portion including a plate-shaped body portion fastened with the body portion insertion grooves of the guide block, and a plurality of micro-needles extending from the plate-shaped body portion, wherein
upon engagement of the guide block, the plate-shaped body portion is fitted in the body portion insertion grooves and fastened with the plate-shaped micro-needle portion, and the drug transfer grooves are engaged with each other to form drug transfer holes,
each of the stepped portions houses micro-needles therein, communicates with the drug discharge holes, and protrudes externally from the bottom of the container,
the micro-needles are passed through the stepped portions to be exposed from lower portions of the stepped portions, and
the drug is discharged through the drug transfer holes to the drug discharge holes along a surface of the micro-needles.

8. The skin drug injector according to claim 1, wherein the injection portion includes:
a container including a plurality of drug discharge holes formed in a bottom; and
a micro-needle block fastened inside the container, wherein
the micro-needle block includes:
a plurality of guide blocks including both sides elongated in a longitudinal direction, drug transfer grooves formed on the both sides and communicating with the plurality of drug discharge holes, body portion insertion grooves formed in the both sides, and a plurality of block protrusions passed through the drug discharge holes to protrude out of the bottom of the container; and
a plate-shaped micro-needle portion including a plate-shaped body portion fastened with the body portion insertion grooves of the guide block, and a plurality of micro-needles extending from the plate-shaped body portion, wherein
upon engagement of the guide block, the plate-shaped body portion is fitted in the body portion insertion grooves and fastened with the plate-shaped micro-needle portion, the micro-needles N are housed in the block protrusions, respectively, the drug transfer grooves are engaged with each other to form drug transfer holes,
the micro-needles are passed through the block protrusions to be exposed from the lower portion, and the drug is discharged through the drug transfer holes along the surface of the micro-needles housed inside the block protrusions.

9. The skin drug injector according to claim 1, wherein the body portion includes an inlet pipe connected to the drug passage hole and protruding into the chamber, and
the inlet pipe allows the drug in the drug bottle to flow into the chamber of the body portion through the drug passage hole.

10. The skin drug injector according to claim 1, wherein the body portion includes a bent ledge formed on an inner side of the enlarged diameter portion, the opening of the enlarged diameter portion is tapered from an end portion toward the bent ledge, and
the container includes a protruding end corresponding to the bent ledge of the enlarged diameter portion.
